(19) United States
(12) Patent Application Publication
(10) Pub. No.: US 2008/0206364 A1
(43) Pub. Date: Aug. 28, 2008

(54) Topical nitric oxide as a treatment of autoimmune diseases

(75) Inventors: Frank J. McCaney, Voorhees, NJ (US); Alex Stenzler, Long Beach, CA (US); Chris Miller, North Vancouver (CA)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Nitric Biotherapeutics, Inc., Bristol, PA (US)

(21) Appl. No.: 11/974,247

(22) Filed: Oct. 12, 2007

Related U.S. Application Data
(60) Provisional application No. 60/851,674, filed on Oct. 13, 2006.

(51) Int. Cl. A61K 33/00; A61P 31/00

(52) U.S. Cl. 424/718

(57) ABSTRACT

The present invention relates to compositions and methods for treatment of a patient affected with an autoimmune disorder, and in an embodiment, a skin-related autoimmune disorder. The treatment involves the application of gaseous nitric oxide to an affected patient, and in an embodiment, to the skin of an affected patient.

### CROSS-REFERENCE TO RELATED APPLICATIONS

The present application is entitled to priority under 35 U.S.C. § 119(e), to U.S. Provisional Application No. 60/851,674 filed on Oct. 13, 2006, which application is hereby incorporated herein by reference in its entirety.

### BACKGROUND OF THE INVENTION

The ability of the immune system to discriminate between “self” and “non-self” antigens is vital to the functioning of the immune system as a specific defense against invading microorganisms. “Non-self” antigens are those antigens on substances entering or present in the body which are detectably different or foreign from the animal's own constituents, whereas “self” antigens are those which, in the healthy animal, are not detectably different or foreign from its own constituents. However, under certain conditions, including in certain disease states, an individual's immune system will identify its own constituents as “non-self,” and initiate an immune response against “self” material, at times causing more damage or discomfort as from an invading microbe or foreign material, and often producing serious illness in an individual. Autoimmune disease results when an individual's immune system attacks his own organs or tissues, producing a clinical condition associated with the destruction of that organ or tissue, as exemplified by diseases such as rheumatoid arthritis, insulin-dependent diabetes mellitus, hemolytic anemias, rheumatic fever, Crohn's disease, Guillain-Barr syndrome, psoriasis, thyroiditis, Graves' disease, myasthenia gravis, glomerulonephritis, autoimmune hepatitis, multiple sclerosis, systemic lupus erythematosus, dystrophic epidermolysis bullosa, and the like. Blocking, neutralizing or inhibiting the immune response or removing its cause in these cases is, therefore, desirable.

Autoimmune disease may be the result of a genetic predisposition alone or as the result of the influence of certain exogenous agents such as, viruses, bacteria, or chemical agents, or as the result of the action of both. Some forms of autoimmunity arise as the result of trauma to an area usually not exposed to lymphocytes, such as neural tissue or the lens of the eye. When the tissues in these areas become exposed to lymphocytes, their surface proteins can act as antigens and trigger the production of antibodies and cellular immune responses which then begin to destroy those tissues. Other autoimmune diseases develop after exposure of the individual to antigens which are antigenically similar to, that is cross-reactive with, the individual's own tissue. For example, in rheumatic fever, an antigen of the streptococcal bacterium which causes rheumatic fever, is cross-reactive with parts of the human heart. The antibodies cannot differentiate between the bacterial antigens and the heart muscle antigens, consequently cells with either of those antigens can be destroyed.

Other autoimmune diseases, for example, insulin-dependent diabetes mellitus (involving the destruction of the insulin producing beta-cells of the islets of Langerhans), multiple sclerosis (involving the destruction of the conducting fibers of the nervous system), and rheumatoid arthritis (involving the destruction of the joint lining tissue), are characterized as being the result of a mostly cell-mediated autoimmune response and appear to be due primarily to the action of T-cells (See, Sinha et al., Science 248:1380 (1990)). Yet others, such as myasthenia gravis and systemic lupus erythematosus, are characterized as being the result of primarily a humoral autoimmune response (Id.). As an example, dystrophic epidermolysis bullosa has been attributed to mutations in the non-collagenous domains of collagen type VII. These mutations result in the lack of formation of the normal anti-parallel collagen type VII dimers. The mutated collagen forms epitopes recognized as “non-self” by the immune system, and therefore autoantibodies are generated, resulting in the rapid degeneration of the basement membrane of the skin (Chen, et al., J. Biol. Chem. 276: 21649 (2001)). Nevertheless, the autoimmune diseases share a common underlying pathogenesis, resulting in the need for safe and effective therapy. Yet none of the presently available drugs are completely effective for the treatment of autoimmune disease, and most are limited by severe toxicity.

Recognition of the important role of cytokines in autoimmune disease has fostered the development of a new generation of therapeutic agents to modulate cytokine activity. Preliminary results of trials in which anti-interferon polyclonal antibodies were administered to a small group of rheumatoid patients suggest improvement in both the clinical and the laboratory manifestations of the disease (Skurkovich et al., Annals of Allergy 39:344-350 (1977)). Moreover, proteins, such as polyclonal antibodies and soluble receptors targeted against interferons and TNF-α are currently being evaluated in clinical trials for the treatment of RA and other autoimmune diseases. The administration of monoclonal antibodies to TNF-α has provided encouraging early results in the treatment of patients with severe RA (Elliott et. al., J. Cell. Biochem., Suppl 17B: 145 (1993); Elliott et al., Lancet 344:1105-1110 (1994)). However, because autoimmune diseases are complex, often characterized by multiple cytokine abnormalities, and having overlapping effects due to the presence of multiple cytokines, effective treatment appears to require the simultaneous administration or utilization of several agents, each targeting a specific cytokine pathway or its by-product.

A separate problem related to conventional treatment of autoimmune diseases of the skin is that the disease may also interfere with the circulation of blood within the affected region (e.g., Raynaud's Syndrome). It is sometimes the case that the disorder causes constriction of the capillaries or other small blood vessels in the affected region, which reduces blood flow. When blood flow is reduced, a lower level of drug is delivered to the affected region. In addition, the affected area of the skin may take a much longer time to heal when blood flow is restricted to the area. This increases the total amount of drug that must be administered to the patient, thereby increasing the cost of using such drugs. Topical agents may sometimes be applied over the affected region. However, topical agents may not penetrate deep within the skin for certain autoimmune diseases which have their genesis in the skin.

In the 1980's, it was discovered by researchers that the endothelium tissue of the human body produced nitric oxide (NO), and that the NO molecule is an endogenous vasodilator, namely an agent that widens the internal diameter of blood vessels. NO, in gaseous form, is most commonly known as an environmental pollutant that is produced as a by product of combustion. At high concentrations, NO is toxic to humans. At low concentrations, researchers have discovered that inhaled NO can be used to treat various pulmonary diseases in patients. For example, NO has been investigated for the treatment of patients with increased airway resistance as a result of emphysema, chronic bronchitis, asthma, adult respiratory distress syndrome (ARDS), and chronic obstructive pulmonary disease (COPD).

NO has also been investigated for its use as a sterilizing agent. It has been discovered that NO will interfere with or kill the growth of bacteria grown in vitro. PCT International Application No. PCT/CA99/01123 published Jun. 2, 2000 discloses a method and apparatus for the treatment of respiratory infections by NO inhalation. NO has been found to have either an inhibitory and/or a cidal effect on pathogenic cells.

While there are few cures for autoimmune disorders, the treatment for many of them includes the exclusion of triggering mechanisms (e.g., stress), avoidance of conditions, medications or substances that can exacerbate the disease (e.g., cold, nicotine, caffeine, etc.) while providing disease specific medications such as vasodilators, calcium channel blockers and anti-inflammatory agents.

McInnes and Liew (1999, Nitric Oxide and Infection, F. Fang, Ed., Plenum Publishers, New York) describe the role of NO in various immune processes in mammals. For example, it is known that NO can induce vasodilatation through relaxation of vascular smooth muscle, and that NO can promote or increase edema. In immune-related inflammatory responses, NO can also modify cellular recruitment.

NO can also modulate T-lymphocyte responsiveness, and can enhance peripheral blood lymphocyte activation (McInnes and Liew, 1999). NO has also been shown to modulate the effects on functional maturation of T lymphocytes. For example, NO can preferentially inhibit Th1 clonal proliferation to antigen. Maturational phenotype, in combination with NO concentration, can influence the modulatory effect of NO on T cells. Furthermore, NO has been implicated in regulation of monokine production.

NO has also been implicated in the modulation of the immune response to infections (McInnes and Liew, 1999). As discussed elsewhere herein, NO can directly act on microbial infections in mammals. NO can also indirectly assist in the eradication of microbial infections through modulation of the immune response. Among the ways that NO can act are through modulation of the Th1 response and through modulation of cytokine levels.

While NO has shown promise with respect to certain medical applications, delivery methods and devices must cope with certain problems inherent in gaseous NO delivery. First, exposure to high concentrations of NO is toxic, especially exposure to NO in concentrations over 1000 ppm. Even lower levels of NO can be harmful if the time of exposure is relatively high. For example, the Occupational Safety and Health Administration (OSHA) has set exposure limits for inhaled NO in the workplace at 25 ppm time-weighted averaged for eight (8) hours. It is extremely important that any device or system for delivering NO include features that prevent the leaking high concentrations of NO into the surrounding environment. If the device is used within a closed space, such as a hospital room or at home, dangerously high levels of NO can build up in a short period of time.

Another problem with the delivery of NO is that NO oxidizes in the presence of oxygen to form NO₂, which is itself toxic, even at low levels. If the delivery device contains an inward leak, unacceptably high levels of NO₂ gas can develop. In addition, to the extent that NO oxidizes to form NO₂, there is less NO available for the desired therapeutic effect. The rate of oxidation of NO to NO₂ is dependent on numerous factors including the concentration of NO, the concentration of O₂, and the time available for reaction. Since NO will react with the oxygen in the air to convert to NO₂, it is desirable to have minimal contact between the NO gas and the outside environment.

Accordingly, there is a need for a device and method for the treatment of autoimmune diseases of the skin by the topical application of NO. The present invention meets these needs.

### BRIEF SUMMARY OF THE INVENTION

The invention includes a method of using topical nitric oxide exposure to treat tissue affected by an autoimmune disorder, comprising providing a source of nitric oxide containing gas, delivering the nitric oxide containing gas to a skin surface containing tissue affected by the autoimmune disorder so as to bathe the tissue affected by the autoimmune disorder with nitric oxide, and optionally, evacuating the nitric oxide containing gas from the area surrounding the tissue affected by the autoimmune disorder.

In an aspect, the autoimmune disorder is a skin-related autoimmune disorder. In an aspect, the skin-related autoimmune disorder is selected from the group consisting of alopecia areata, psoriasis, vitiligo, epidermolysis bullosa, scleroderma, antiphospholipid syndrome, Sjorgren's Syndrome and Raynaud's Syndrome.

In an aspect, the nitric oxide is delivered to the skin surface using a bathing unit surrounding the tissue affected by the autoimmune disorder. In an aspect, the bathing unit comprises one or more openings for the escape of gas. In an aspect, the concentration of nitric oxide in the nitric oxide containing gas that bathes the tissue affected by the autoimmune disorder is equal to or less than about 300 ppm. In an aspect, the method includes refreshing the tissue affected by the autoimmune disorder with a fresh supply of nitric oxide containing gas, optionally using an agitator. In an aspect, a jet of nitric oxide containing gas is delivered to the portion of the skin surface containing the tissue affected by the autoimmune disorder. In an aspect, the source of nitric oxide containing gas is selected from the group consisting of a pressurized cylinder containing nitric oxide gas and direct production of nitric oxide gas.

In an aspect, a method of the invention further comprises at least one of the steps selected from the group consisting of monitoring the concentration of nitric oxide bathing the tissue affected by the autoimmune disorder, monitoring the concentration of nitrogen dioxide bathing the tissue affected by the autoimmune disorder, stripping nitric oxide from the evacuated gas, and stripping nitrogen dioxide from the evacuated gas.

The invention includes a method of treating skin tissue affected by a skin-related autoimmune disorder comprising the steps of identifying the affected skin tissue of a human, providing a flow-controlled source of nitric oxide gas, and delivering the nitric oxide gas to at least a portion of the affected skin tissue.

In an aspect, the invention including sealing the device comprising the nitric oxide gas to prevent contact with air in the atmosphere. In an aspect, a method includes diluting the nitric oxide gas with at least one of the members selected from the group consisting of air and an inert gas. In an aspect, the flow-controlled source of nitric oxide gas flows from a pressurized cylinder containing nitric oxide gas. In an aspect, the pressure of the nitric oxide gas is reduced before delivering the nitric oxide gas to the affected skin tissue. In an aspect, the nitric oxide gas is evacuated at a flow rate substantially equal to a flow rate of the nitric oxide gas delivered to the affected skin tissue.

The invention includes a method of treating skin tissue affected by an autoimmune disorder with topical exposure to nitric oxide gas comprising the steps of providing a source of nitric oxide gas. diluting the nitric oxide gas with at least one of the members selected from the group consisting of air and an inert gas, and delivering the diluted nitric oxide gas to at least a portion of the affected skin tissue.

In an aspect, diluting the nitric oxide gas is performed through a gas blender. In an aspect, the nitric oxide gas delivered to the skin is flow controlled. In an aspect, the step of diluting the nitric oxide gas further comprises the step of delivering the inert gas to a substantially air-tight sealed area over the affected skin tissue. In an aspect, the invention includes the step of monitoring the concentration of nitric oxide being delivered.

The invention includes a method of alleviating Alopecia areata-mediated loss of hair on a skin surface, comprising providing a source of nitric oxide containing gas, and delivering nitric oxide containing gas to the skin surface which is afflicted with hair loss so as to bathe the skin surface with nitric oxide, thereby alleviating the loss of hair from the skin surface.

The invention includes a method of alleviating autoimmune-mediated scarring of a skin surface, wherein the scarring is a result of excess collagen deposition, comprising providing a source of nitric oxide containing gas, and delivering nitric oxide containing gas to the skin surface which is afflicted with scarring so as to bathe the skin surface with nitric oxide, thereby alleviating scarring of the skin surface.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 illustrates a schematic representation of the NO delivery device according to one aspect of the invention.

FIG. 2 illustrates a bathing unit surrounding the foot of a patient.

FIG. 3 illustrates a bathing unit surrounding the hand of a patient.

FIG. 4 illustrates a bathing unit including an agitator located therein.

FIG. 5 illustrates histology analysis of full thickness infected wound exposed to 200 ppm gNO for 24 hours.

FIG. 6 shows mRNA expression for collagen and collagenase following exposure to 200 ppm gNO for 24 hours and 48 hours.

FIG. 7 illustrates the morphology of fibroblast cells exposed inside gNO chamber to less than 200 ppm NO versus control group inside conventional tissue culture incubator.

FIG. 8 illustrates increase in fibroblast cell proliferation following exposure to 200 ppm of NO in comparison with control.

FIG. 9 illustrates cell attachment capacity of human fibroblasts following exposure to 160 ppm of gNO.

FIG. 10 shows the results of fibroblasts grown in a 3D matrix and exposed to 200 ppm NO for 8 hours per day for 3 days compared with control cells in air or conventional incubator.

FIG. 11 shows the amount of proliferation of fibroblasts grown in a 3D matrix and exposed to 200 ppm NO for 8 hours per day for 3 days compared with control cells in air or conventional incubator.

FIG. 12 shows the amount of tube formation in human endothelial cells grown in matrigel and exposed to air (top panels) or 200 ppm NO (bottom panels) for 24 hours. Left panels at 8 hours of exposure. Right panels at 24 hours of exposure.

FIG. 13 shows increased collagen mRNA expression in fibroblast exposed to 5 ppm of NO.

FIG. 14 is an image of a local wound cover that uses just a single tube to flow the gas into the dressing and allows the gas to exit through one or more holes in the top occlusive cover.

FIG. 15, comprising FIGS. 15A-15C, is a series of images illustrating the healing of a leg ulcer induced by an autoimmune disorder. FIG. 15A illustrates the injury prior to treatment, FIG. 15B illustrates the injury after preliminary treatment according to the invention, and FIG. 15C illustrates the injury after continued treatment according to the invention.

### DETAILED DESCRIPTION

The present invention relates to compositions and methods for the treatment of skin-related autoimmune disorders by using nitric oxide (“NO”). In particular, the invention provides a method of treating a skin-related autoimmune disorder by topically applying NO to the skin of an affected patient. This is because it is shown herein that topical administration of NO can be used to directly treat an autoimmune disorder directly affecting the skin, or to treat an autoimmune disorder which involves the skin indirectly, wherein the effect on the skin is a non-primary effect of the autoimmune disorder.

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

The term “autoimmune disease” as used herein is defined as a disorder that results from an autoimmune response. An autoimmune disease is the result of an inappropriate and excessive response to a self-antigen. Examples of autoimmune diseases include but are not limited to, Addison's disease, alopecia areata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

As used herein, an autoimmune disease that can be treated according to the present invention includes any autoimmune disease that exists at any site accessible to the application of NO gas. Such sites include, but are not limited to, conditions existing on the skin surface, conditions existing under the skin surface (including blood vessels and subdermal tissues), conditions existing in openings or crevices of the surface of the body, portions of the body accessible through the respiratory and/or gastrointestinal tracts, and conditions exposed due to opening of any part of the body due to a surgical procedure. Other examples of such sites include the eye, mouth, mucous membranes, and blood vessels.

As used herein, the term “skin-related autoimmune disorder” refers to an autoimmune disorder that either partially or wholly affects the skin tissue of a mammal afflicted with the autoimmune disorder. A “skin-related autoimmune disorder” may be an autoimmune disorder directly affecting the skin (e.g., psoriasis), or it may be an autoimmune disorder that has a non-primary affect on the skin (e.g., autoimmune inner ear disease). A skin-related autoimmune disorder directly affecting the skin may also be referred to as an “autoimmune disorder of the skin.”

As used herein, the term “modulate” is meant to refer to any change in biological state, i.e. increasing, decreasing, and the like.

The term “T-cell” as used herein is defined as a thymus-derived cell that participates in a variety of cell-mediated immune reactions.

As used herein, a “therapeutically effective amount” is the amount of a therapeutic composition sufficient to provide a beneficial effect to a mammal to which the composition is administered.

The terms “patient” and “individual” are interchangeably used to mean a warm-blooded animal, such as a mammal, suffering from a disease, such as a skin-related autoimmune disease. It is understood that humans and animals are included within the scope of the term “patient” or “individual.”

The term “treat” or “treatment,” as used herein, refers to the alleviation (i.e., “diminution”) and/or the elimination of a symptom or a source of a given disease. By way of several non-limiting examples, a symptom of a skin-related autoimmune disorder can be treated by alleviating the symptom of that disorder. A symptom of a skin-related autoimmune disorder can also be treated by altogether eliminating the symptom of that disorder. A skin-related autoimmune disorder can be treated by alleviating the source, or “cause,” of that disorder. A skin-related autoimmune disorder can also be treated by altogether eliminating the source of that disorder.

“Evacuating” as the term is used herein, refers to the partial or complete removal of a substance from a specific region or area. By way of two separate non-limiting examples according to the present invention, nitric oxide gas can be partially removed from the area under an enclosed device which surrounds a portion of the skin of a patient, or nitric oxide gas can be completely removed from the area under an enclosed device which surrounds a portion of the skin of a patient. In these examples, the nitric oxide gas may or may not be replaced with a “substitute” gas. The “substitute” gas, according to the invention, may be any gas, including nitric oxide gas.

### DESCRIPTION

The present invention provides a method of treating various autoimmune diseases and disorders, by applying gaseous nitric oxide to a patient having the disease or disorder. In one aspect, the invention provides a method of treating autoimmune diseases and disorders by applying gaseous nitric oxide to the skin of a patient having the disease or disorder.

The present invention also provides a method of treating various skin-related autoimmune diseases and disorders, including, but not limited to, pemphigus vulgaris, alopecia areata, vitiligo, dystrophic epidermolysis bullosa and psoriasis, by applying gaseous nitric oxide to the skin of a patient having the disease or disorder. The invention also encompasses other autoimmune disorders, wherein the autoimmune disorder affects the skin. That is, the invention includes autoimmune disorders that do not necessarily originate with the skin, but nonetheless, somehow affect the skin adversely. By way of a non-limiting example, such disorders include autoimmune inner ear disease, as well as antiphospholipid syndrome, scleroderma, and Sjorgren's Syndrome.

Alopecia areata is an autoimmune disorder wherein the patient's hair follicles are attacked by the immune system resulting in hair loss and arrest of hair growth. The disease affects hair follicles over the entire body, including the scalp. The disease is characterized by small, smooth bald patches, usually on the scalp, and can progress to total baldness.

Vitiligo is a skin-related autoimmune disorder that affects the pigmentation of the skin. The immune system of a patient suffering from vitiligo attacks the patient's melanocytes, the pigment-producing cells of the body, resulting in hypopigmented skin. Vitiligo usually affects the chest and abdomen, but hypopigmentation around the mouth, nostrils, and eyes also occurs. The resulting hypopigmentation is more noticeable in populations normally having darker pigmented skin, but the disease occurs in all populations. Vitiligo often occurs in people with insulin-dependent diabetes mellitus.

Psoriasis is a chronic skin disease characterized by periodic flare-ups of a scaly rash, often reddish in color. The disease usually targets elbows, knees, scalp, ears, and the lower back. Fingernails and toenails are also affected. Approximately ten to fifteen percent of people afflicted with psoriasis will develop inflammatory arthritis, suggesting a link between these diseases.

Dystrophic epidermolysis bullosa is an autoimmune-mediated skin disease characterized by widespread blistering and scarring on the skin and mucous membranes. Dystrophic epidermolysis bullosa usually manifests as generalized blistering, absent of dystrophic finger- and toenail development, skin cysts, scarring, anemia due to blood loss resulting from blistering, growth retardation, dental caries, gastrointestinal and genitourinary tract blistering, and fusion of the fingers and toes. Dystrophic epidermolysis bullosa usually results in death during childhood due to anemia, septicemia, malnutrition, or other complications.

Pemphigus vulgaris is an autoimmune skin disorder characterized by blistering and loss of cell-cell adhesion (acantholysis) of all skin surfaces, especially the mucous membranes. Pemphigus vulgaris often presents with oral lesions, and then progresses to generalized mucosal lesions followed by generalized skin lesions. Secondary infections in affected areas are common, and malignancies have been reported as well. Before the advent of corticosteroids, the mortality rate of patients with pemphigus vulgaris was 100%, but with steroid therapy, the mortality rate has dropped significantly to about 5-15%. Regression is common, even with active therapy.

Aside from the importance of treating the autoimmune nature of the aforementioned diseases, treatment of these diseases is important to improving social interactions of patients afflicted with any one of these diseases. Patients having a skin-related autoimmune disease, such as alopecia areata, vitiligo, dystrophic epidermolysis bullosa, pemphigus vulgaris or psoriasis, are particularly sensitive to the consequences of the disease since the disease affects the patient's outward appearance. It is suggested that people, especially children, afflicted with any of the skin-related autoimmune diseases may feel awkward, and even lack self-esteem in a social situation due to their appearance. Thus treatment of these diseases, while aiding in preventing autoimmune reactions, also may improve the patient's emotional status.

Nitric oxide as a vascular relaxing agent, has the potential to treat autoimmune disorders of the skin that affect blood flow. By way of a non-limiting example, Raynaud's phenomenon is an autoimmune disease with symptoms that include significant reduction in blood flow to the small arteries and capillaries of the fingers. This results in a sensation of cold, numbness and pain in the peripheral limbs. Topical nitric oxide may be used to provide adequate vasodilatation to increase blood flow and reduce the patient symptoms. The invention should also be understood to be useful to treat any autoimmune disorder that affects blood flow, wherein the disorder can be treated by the administration of gaseous NO.

Other autoimmune diseases exhibit symptoms of excessive collagen formation, such as in scleroderma. The upregulation of collagenase, elicited by administration of nitric oxide, has the potential to reduce the thickening of the skin in patients having such autoimmune diseases.

Several autoimmune diseases have the effect of compromising circulation or their inflammatory processes, and consequently, increase the occurrence of opportunistic infections. While the infection is not directly a function of the disease, there is a causal relationship between the disease and the infection, and nitric oxide can be used according to the present invention as an important adjunctive therapy for the treatment of such infections. Furthermore, the present invention can be used to provide a therapeutic benefit to the lesion by providing direct anti-inflammatory and/or anti-immune response action.

Therefore, in one embodiment, the present invention provides a method of alleviating the symptoms of an autoimmune disorder by topically applying NO gas to the affected patient. In another embodiment, the present invention provides a method of alleviating the symptoms of a skin-related autoimmune disorder by topically applying NO gas to the affected skin of a patient. In yet another embodiment, the present invention provides a method of eliminating the symptoms of a skin-related autoimmune disorder by topically applying NO gas to the affected skin of a patient. By way of a non-limiting example, the symptoms of psoriasis can be treated by directly reducing the severity of the symptoms of the psoriatic condition in skin of the patient. By way of another non-limiting example, the symptoms of psoriasis can be treated by directly reducing the source of the presented symptoms of the psoriatic condition in skin of the patient. That is, the autoimmune attack resulting in the psoriatic symptoms can be diminished by treatment of the affected skin of a patient with gaseous NO.

By the term “affected skin” is meant regions of skin that exhibit lesions or other signs and symptoms of the subject autoimmune disease. However, the term should not be construed to include only those regions of skin that exhibit overt evidence of disease, but rather should also be construed to include regions of the skin that may be asymptomatic, i.e., that do not contain overt lesions or other signs of disease, but that may be affected nonetheless and that may in time exhibit more overt evidence of disease.

In yet another embodiment, the present invention provides a method of alleviating the source of the symptoms of a skin-related autoimmune disorder by topically applying NO gas to the affected skin of a patient. In another embodiment, the invention provides a method of eliminating the source of the symptoms of a skin-related autoimmune disorder by topically applying NO gas to the affected skin of a patient. By way of a non-limiting example, the symptoms of psoriasis can be treated by directly eliminating the source of the presented symptoms of the psoriatic condition in skin of the patient. That is, the autoimmune attack resulting in the psoriatic symptoms can be eliminated by treatment of the affected skin of a patient with gaseous NO.

Typically, gaseous NO is administered to the skin of a patient using a device for delivery of NO to the skin. The device is preferably leak proof to the largest extent possible to avoid any chance of a potentially a dangerous build up of NO and NO₂ concentrations. In addition, the device should be capable of delivering NO to the affected region of the patient without allowing the introduction of air that would otherwise react with NO to produce NO₂. The application of NO to the affected region preferably decreases the time required to heal the affected area by reducing any associated bacterial burden, improving blood supply to the tissues, reducing inflammation, and when indicated as described elsewhere herein, modulating the production of collagen and collagenase.

In an aspect of the invention, the application of NO to the affected region decreases the time required to heal the affected area, induces healing of the affected area, or directly contributes to the healing of the area. In an embodiment of the invention, application of NO to the affected region acts by two or more different mechanisms to promote healing.

In an aspect of the invention, improvement of symptoms in a patient being treated with a composition of the invention, method of the invention, or both, the promotion of healing can be measured by any one of many ways known in the art. Generally, promotion of healing can be ascertained by an improvement, or a “lessening,” of the symptoms of the disease or disorder, upon treatment with nitric oxide according to a method and/or composition of the invention.

By way of a non-limiting example, thermal imaging can be used to ascertain an improvement of one or more symptoms. By way of another non-limiting example, the improvement of psoriasis can be evaluated by a clearing of a psoriatic rash, among other indicators.

While the methods of the invention should not be construed to be limited solely to the mechanisms and devices described herein for delivery of NO to skin, certain embodiments encompassing various mechanisms and devices are described herein as examples of how to perform the methods of the invention. It is understood that the methods of the invention may be practiced using other known or heretofore unknown mechanisms and devices and the invention should be construed to encompass all such mechanisms and devices as appropriate.

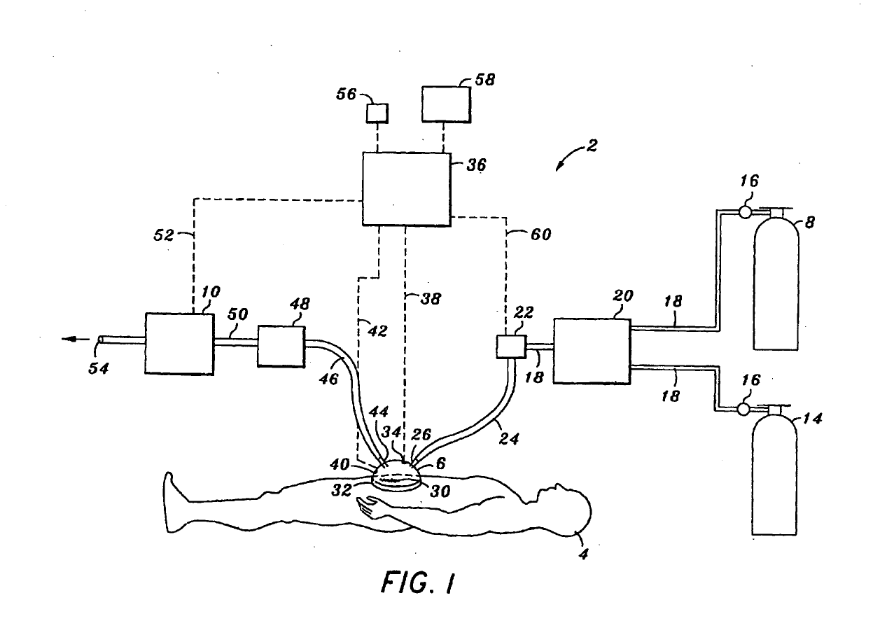

In one set of embodiments and referring now to FIG. 1 herein, an NO delivery device 2 is shown connected to a patient 4. In its most general sense, the NO delivery device 2 includes a bathing unit 6 that is fluidically connected to a NO gas source 8, a flow control valve 22, and a vacuum unit 10. FIG. 1 therefore illustrates one preferred embodiment of the device useful for administration of NO to a patient in the methods of the invention.

In FIG. 1, the NO gas source 8 is a pressurized cylinder containing NO gas. While the use of a pressurized cylinder is the preferred method of storing the NO-containing gas source 8, other storage and delivery means, such as a dedicated feed line (wall supply), or direct production of NO gas, can also be used. Typically, the NO gas source 8 is a mixture of N₂ and NO. While N₂ is typically used to dilute the concentration of NO within the pressurized cylinder, air, or any inert gas can also be used, alone or in combination. When the NO gas source 8 is stored in a pressurized cylinder, it is preferable that the concentration of NO in the pressurized cylinder fall within the range of about 800 ppm to about 20,000 ppm. Commercial nitric oxide manufacturers typically produce nitric oxide mixtures for medical use at around the 1000 ppm range. Extremely high concentrations of NO are undesirable because accidental leakage of NO gas is more hazardous, and high partial pressures of NO tends to cause the spontaneous degradation of NO into nitrogen dioxide. Pressurized cylinders containing low concentrations of NO (i.e., less than 100 ppm NO) can also be used in accordance the device and method disclosed herein. Of course, the lower the concentration of NO used, the more often the pressurized cylinders will need replacement.

FIG. 1 also shows source of dilutent gas 14 as part of the NO delivery device 2 that is used to dilute the concentration of NO. The source of dilutent gas 14 can contain N₂, O₂, air, an inert gas, or a mixture of these gases. It is preferable to use a gas such as N₂ or an inert gas to dilute the NO concentration since these gases will not oxidize the NO into NO₂ as would O₂ or air. The source of dilutent gas 14 is shown as being stored within a pressurized cylinder. While the use of a pressurized cylinder is shown in FIG. 1 as the means for storing the source of dilutent gas 14, other storage and delivery means, such as a dedicated feed line (wall supply) or pumps can also be used.

The NO gas from the NO gas source 8 and the dilutent gas from the dilutent gas source 14 preferably pass through pressure regulators 16 to reduce the pressure of gas that is admitted to the NO delivery device 2. The respective gas streams pass via tubing 18 to an optional gas blender 20. The gas blender 20 mixes the NO gas and the dilutent gas to produce a NO-containing gas that has a reduced concentration of NO. In an embodiment, the NO-containing gas that is output from the gas blender 20 has a concentration of NO that is about 300 ppm. Preferably, the NO-containing gas that is output from the gas blender 20 has a concentration of NO that is less than about 300 ppm. Even more preferably, the concentration of NO-containing gas that is output from the gas blender 20 is less than about 200 ppm NO. Even more preferably, the concentration of NO-containing gas that is output from the gas blender 20 is less than about 100 ppm NO.

In an aspect, the NO concentration in the gas used for treatment can fall in the range of 25 ppm to 10,000 ppm. Based on the disclosure set forth herein, the skilled artisan will understand how to adjust the concentration of NO, and moreover, how to selected the concentration of NO necessary for any particular application. By way of a non-limiting example, a higher concentration of NO may be used when a shorter treatment time is desired. However, it will be understood that the present application also teaches the skilled artisan how to determine the concentration of NO useful for any particular set of circumstances, based on the time of treatment and also based on the desired outcome of the treatment (e.g., alleviation of symptoms versus eradication of disease).

The NO-containing gas that is output from the gas blender 20 travels via tubing 18 to a flow control valve 22. The flow control valve 22 can include, for example, a proportional control valve that opens (or closes) in a progressively increasing (or decreasing if closing) manner. As another example, the flow control valve 22 can include a mass flow controller. The flow control valve 22 controls the flow rate of the NO-containing gas that is input to the bathing unit 6. The NO-containing gas leaves the flow control valve 22 via flexible tubing 24. The flexible tubing 24 attaches to an inlet 26 in the bathing unit 6. The inlet 26 might include an optional one way valve 64 (see FIG. 3) that prevents the backflow of gas into the tubing 24.

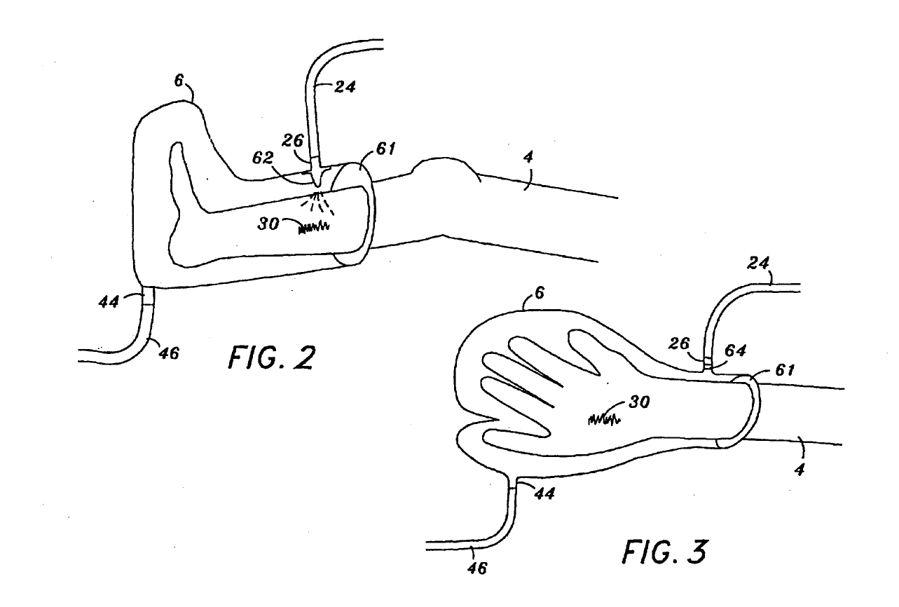

Still referring to FIG. 1, the bathing unit 6 is shown sealed against the skin surface of a patient 4. The affected area 30 is enclosed by the bathing unit 6. The bathing unit 6 preferably includes a seal portion 32 that forms a substantially air-tight seal with the skin of the patient 4. Substantially air-tight is meant to indicate that the NO-containing gas does not leak out of the bathing unit 6 in significant amounts. The seal portion 32 may comprise an inflatable seal 61, such as that shown in FIGS. 2 and 3, or alternatively the seal portion 32 may comprise a flexible skirt or the like that confirms to the surface of the patient 4. The seal portion 32 also might include an adhesive portion that adheres to the skin surface of a patient 4. In other envisioned embodiments, the sealing portion 32 may merely comprise the interface of the bathing unit 6 with the surface of the patient's 4 skin.

The bathing unit 6 can be made of a virtually limitless number of shapes and materials depending on its intended use. The bathing unit 6 might be formed as a rigid structure, such as that shown in FIG. 1, that is placed over an affected area 30. Alternatively, the bathing unit 6 can be formed of a flexible, baglike material that is inflatable over an affected area 30. FIG. 2 shows such a structure in the shape of a boot that is placed over the patient's 4 foot. FIG. 3 shows another inflatable bathing unit 6 that is formed in the shape of a mitten or glove that is worn over the patient's 4 hand.

In one preferred embodiment of the invention, the bathing unit 6 includes an NO sensor 34 that measures the concentration of NO gas within the bathing unit 6. The NO sensor 34 preferably reports this information to a controller 36 via signal line 38. An optional NO₂ sensor 40 can also be included within the bathing unit 6. The NO₂ sensor 40 preferably reports the concentration of NO₂ to the controller 36 via signal line 42. The sensors 40, 42 can be a chemilluminesense-type, electrochemical cell-type, or spectrophotometric-type sensor.

The bathing unit 6 also includes an outlet 44 that is used to remove gas from the bathing unit 6. The outlet 44 is preferably located away from the gas inlet 26 such that NO gas does not quickly enter and exit the bathing unit 6. Preferably, the inlet 26 and outlet 44 are located in areas of the bathing unit 6 such that the NO gas has a relatively long residence time. Flexible tubing 46 is connected to the outlet 44 and provides a conduit for the removal of gases from the bathing unit 6.

In one preferred embodiment of the invention, the flexible tubing 46 is in fluid communication with an absorber unit 48. The absorber unit 48 preferably absorbs or strips NO from the gas stream that is exhausted from the bathing unit 6. It is also preferable for the absorber unit 48 to also absorb or strip NO₂ from the gas stream that is exhausted from the bathing unit 6. Since these gases are toxic at high levels, it is preferable that these components are removed from the delivery device 2 prior to the gas being vented to the atmosphere. In addition, these gases can react with the internal components of the vacuum unit 10 and interfere with the operation of the delivery device 2.

The now clean gas travels from the absorbing unit 48 to the vacuum unit 10 via tubing 50. The vacuum unit 10 provides a negative pressure within the tubing 50 so as to extract gases from the bathing unit 6. The vacuum unit 10 is preferably controllable with respect to the level of vacuum or suction supplied to the tubing 50 and bathing unit 6. In this regard, in conjunction with the flow control valve 22, the amount of NO gas within the bathing unit 6 can be regulated. Preferably, the vacuum unit 10 is coupled with the controller 36 via a signal line 52. The controller 36, as discussed below, preferably controls the level of output of the vacuum unit 10. The gas then passes from the vacuum unit 10 to a vent 54 that is open to the atmosphere.

It should be understood that the absorbing unit 48 is an optional component of the delivery device 2. The gas laden with NO and NO₂ does not have to be removed from the gas stream if there is no concern with local levels of NO and NO₂. For example, the gas can be exhausted to the outside environment where high concentrations of NO and NO₂ will not develop. Alternatively, a recirculation system can be used to recycle NO with the bathing unit 6.

Still referring to FIG. 1, the delivery device 2 preferably includes a controller 36 that is capable of controlling the flow control valve 22 and the vacuum unit 10. The controller 36 is preferably a microprocessor-based controller 36 that is connected to an input device 56. The input device 56 is used by an operator to adjust various parameters of the delivery device such as NO concentration, residence time of NO, pressure within the bathing unit 6, etc. An optional display 58 can also be connected with the controller 36 to display measured parameters and settings such as the set-point NO concentration, the concentration of NO within the bathing unit 6, the concentration of NO₂ within the bathing unit 6, the flow rate of gas into the bathing unit 6, the flow rate of gas out of the bathing unit 6, the total time of delivery, and the like.

The controller 36 preferably receives signals from sensors 34, 40 regarding gas concentrations if such sensors 34, 40 are present within the delivery device 2. Signal lines 60, 52 are connected to the flow control valve 22 and vacuum unit 10 respectively for the delivery and receipt of control signals.

In another embodiment of the invention, the controller 36 is eliminated entirely. In this regard, the flow rate of the gas into the bathing unit 6 and the flow rate of the gas out of the bathing unit 6 are pre-set or adjusted manually. For example, an operator can set a vacuum output that is substantially equal to the flow rate of the gas delivered to the bathing unit 6 via the flow control valve 22. In this manner, NO gas will be able to bathe the affected area 30 without any build-up or leaking of NO or NO₂ gas from the delivery device 2.

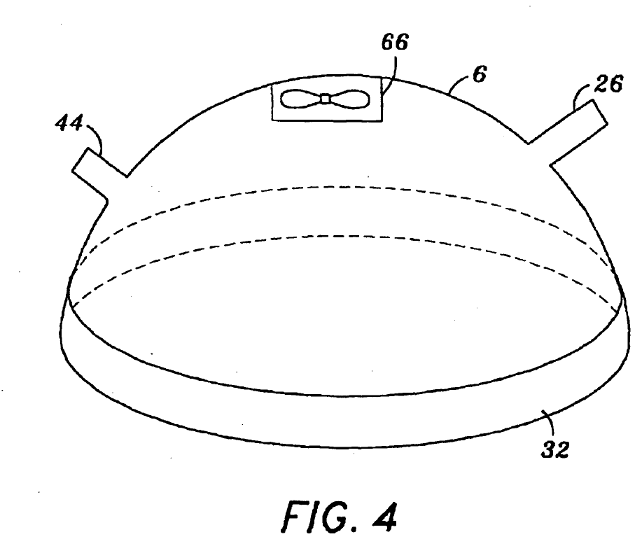

FIG. 2 illustrates a bathing unit 6 in the shape of a boot that is used to treat an affected area 30 located on the leg of the patient 4. The bathing unit 6 includes an inflatable seal 61 that surrounds the leg region to make a substantially air-tight seal with the skin of the patient 4. This embodiment shows a nozzle 62 that is affixed near the inlet 26 of the bathing unit 6. The nozzle 62 directs a jet of NO gas onto the affected area 30. The jet of gaseous NO aids in penetrating the affected area 30 with NO to treat the skin affected by an autoimmune disorder. FIG. 3 shows another embodiment of the bathing unit 6 in the shape of a mitten or glove. The bathing unit 6 is also inflatable and contains an inflatable seal 61 that forms a substantially air-tight seal around the skin of the patient 4. FIG. 3 also shows an optional one way valve 64 located in the inlet 26. As seen in FIGS. 3 and 4, the inlet 26 and outlet 44 are located away from one another, and preferably on opposing sides of the treated area such that freshly delivered NO gas is not prematurely withdrawn from the bathing unit 6.

For treatment of an affected area 30, the bathing unit 6 is placed over the affected area 30. An air-tight seal is then formed between the skin of the patient 4 and the bathing unit 6. If the bathing unit 6 has an inflatable construction, the bathing unit 6 must be inflated with gas. Preferably, the bathing unit 6 is initially inflated only with the dilutent gas to prevent the leaking of NO and NO₂ from the device 2. Once an adequate seal has been established, the operator of the device initiates the flow of NO from the NO gas source 8 to the bathing unit 6. As described above, this may be accomplished manually or via the controller 36. The skilled artisan will know how to establish the appropriate seal—either air-tight, or less than air-tight (e.g., “free-flowing”)—depending upon the particular objective and goal for treatment under any particular set of facts.

Once the bathing unit 6 has started to fill with NO gas, the vacuum unit 10 is turned on and adjusted to the appropriate output level. For an inflatable bathing unit 6, the output level (i.e., flow rate) of the vacuum unit 10 should be less than or equal to the flow rate of NO gas entering the bathing unit 6 to avoid deflating the bathing unit 6. In embodiments of the device where the bathing unit 6 is rigid, the vacuum unit 10 can be set to create a partial vacuum within the bathing unit 4. In this regard, the partial vacuum helps to form the air-tight seal between the skin of the patient 4 and the bathing unit 6. Of course, the vacuum unit 10 can also be set to withdraw gas at a substantially equal rate as the gas is delivered to the bathing unit 6. An effective amount of NO is delivered to the bathing unit 6 to alleviate or eliminate the effects of the autoimmune disorder of the skin. In another embodiment of the invention, an effective amount of NO is delivered to the bathing unit 6 to alleviate or eliminate the source of the autoimmune disorder of the skin.

FIG. 4 shows another embodiment of the invention in which the bathing unit 6 includes an agitator 66 that is used to create turbulent conditions inside the bathing unit 6. The agitator 66 preferably is a fan-type of mechanism but can include other means of creating turbulent conditions within the bathing unit 6. The agitator 66 aids in refreshing the affected area 30 with a fresh supply of NO gas.

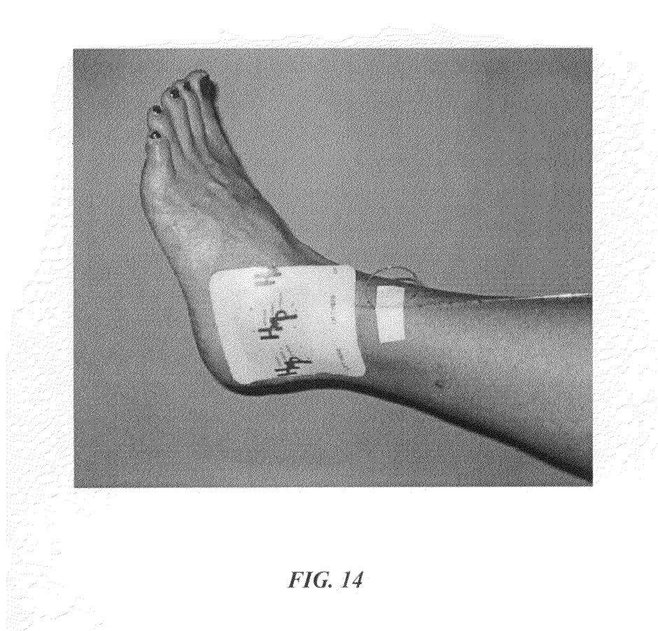

FIG. 14 illustrates another embodiment of the invention, in which the bathing unit comprises a wound cover that has one or more openings for the release of NO gas. In an embodiment, NO gas is introduced underneath the bathing unit, such that the gaseous NO is largely contained beneath the unit, in a space between the unit and the skin. However, the NO gas and spent NO gas are free to escape through the one or more openings in the bathing unit.

As will be understood by the skilled artisan, when armed with the present disclosure, variations in the number and/or size of the openings in such a bathing unit can be used to regulate the pressure of NO gas underneath the wound cover, or to regulate the amount of time that the gaseous NO is in contact with the skin beneath the wound cover. It will also be understood that in any method or apparatus of the invention, the NO gas that is delivered to a patient may optionally be evacuated after use. The skilled artisan will understand, when armed with the disclosure set forth herein, how to determine the timing and method of evacuation.

### EXAMPLES

The invention is now described with reference to the following Examples. These Examples are provided for the purpose of illustration only, and the invention is not limited to these Examples, but rather encompasses all variations which are evident as a result of the teachings provided herein.

### Experimental Example 1

### Uses and Applications of Nitric Oxide According to the Invention

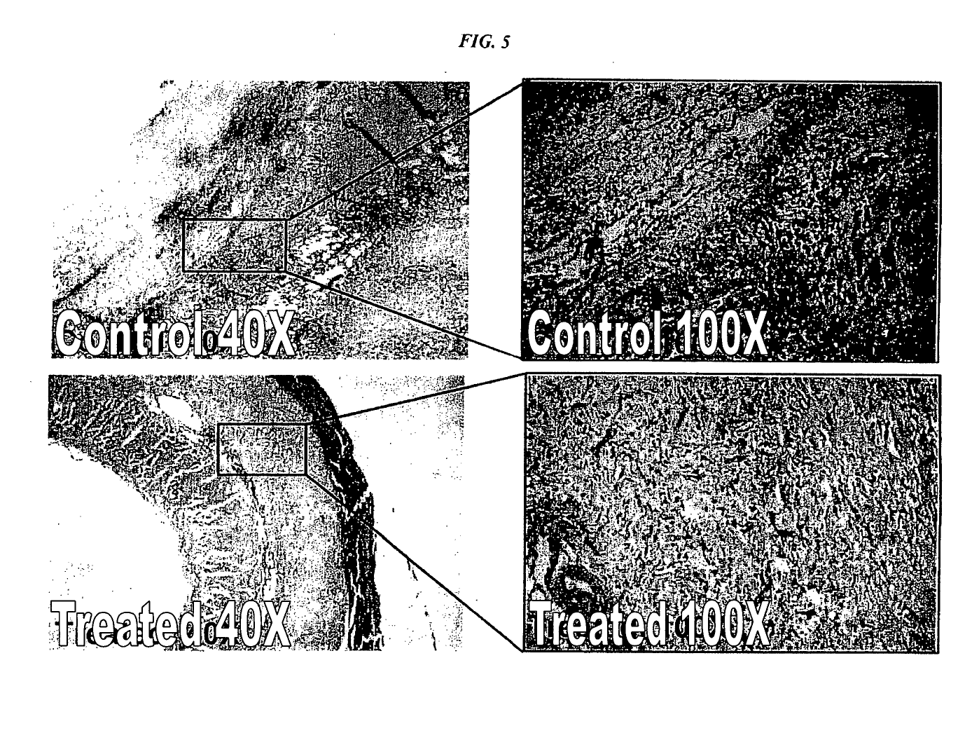

FIG. 5 presents histological analysis of tissue blocks prepared on wound punch biopsies from animals in NO-treated and control groups. Samples from the control group show more advanced neutrophil infiltration and so a higher degree of inflammatory reaction. A lower level of neutrophil concentration is seen in wounds treated with gNO. Wounds treated with gNO also show a layer of granular tissue closing on the wound, but control wounds remain open for longer period of time. Overall, a healthier healing process is observed in the wounds treated with gNO. No toxic effects (cellular debris) can be seen in gNO treated group.

While the inflammatory response is integral to wound healing, an aberrant inflammatory response is believed to be one causal factor in chronic wounds and excess exudate. NO inhibits platelet aggregation, assists in maintaining vascular tone, and inhibits mast cell degranulation (Delledonne M, et al., (2003) The functions of nitric oxide-mediated signaling and changes in gene expression during the hypersensitive response, Antioxid Redox Signal, 5:33-41; and Hickey M J., (2001), Role of inducible nitric oxide synthase in the regulation of leukocyte recruitment, Clin Sci (Lond), 100:1-12). NO produced constitutively by endothelial cells has been shown to have an on-going anti-inflammatory effect. This may in part be due to its effect on platelet aggregation. iNOS is upregulated during the inflammatory response. Studies have shown that iNOS derived NO may also have anti-inflammatory characteristics. Id. Collectively, by maintaining vascular tone, promoting angiogenesis, moderating inflammation and inhibiting mast cell degranulation, NO can be viewed as an important molecule for exudate management. Accordingly, exogenously applied nitric oxide may duplicate and supplement the actions of endogenous nitric oxide to reduce the local inflammatory response as well as down regulate the message that the systemic inflammatory response system had been receiving to increase the sending of inflammatory cells. This eventually may lead to a healthy level of exudate production.

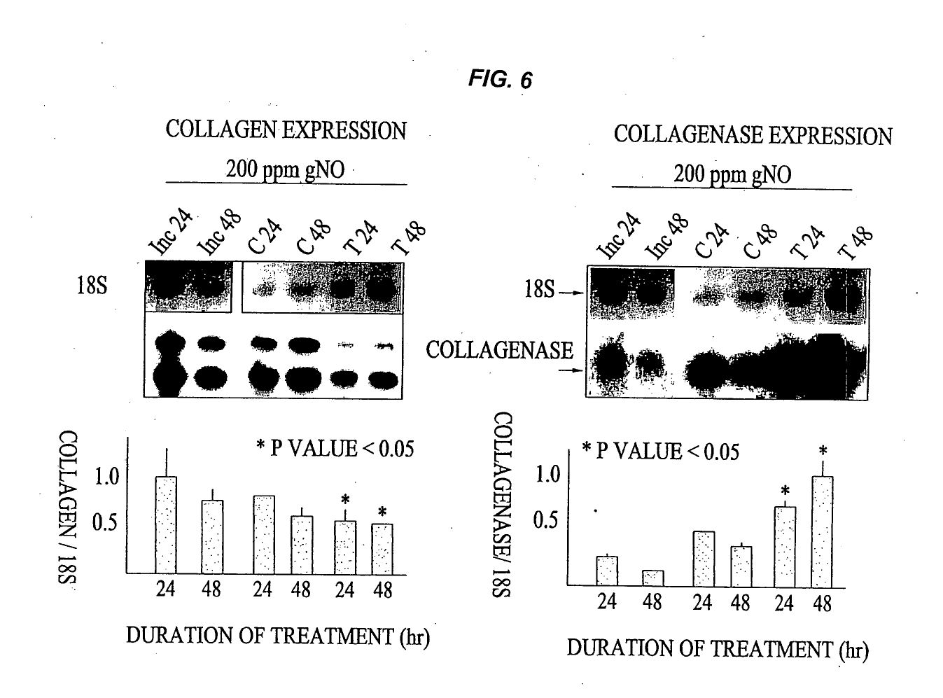

FIG. 6 shows that expression of collagenase mRNA is increased as the exposure time to high concentration of gNO (at 200 ppm) increases. This suggests that high concentration of nitric oxide upregulates collagenase, which may lead to the enzymatic cleavage of collagen. An independent study by Witte et al (2002) found that MMP-2 activity was also upregulated by NO donors. Witte M B, et al, (2002) Nitric oxide enhances investigational wound healing in diabetes, Br J Surg., 89:1594-601. Thus, Applicants believe that NO may upregulate expression of both collagenase (MMP-1) and gelatinase (MMP-2), which may be important in keeping the wound clean from necrotic tissue while not prolonging the inflammatory phase.

Rather than applying exogenous collagenase for enzymatic debridement of necrotic tissue, exposing a wound with necrotic tissue to exogenous NO gas to upregulate endogenous collagenase may be more beneficial. When endogenous collagenase is released by the cell, it automatically releases TIMP's (tissue inhibitor of metalloproteinase). This ensures that the matrix degradation is coordinated and allows the establishment of sharp geographical boundaries of collagenolytic activity and the protection of areas of connective tissue from the activity of the enzyme. In contrast, use of exogenous collagenase material to debride a wound confers no protection to specific areas of the wound as it is active on every cell that comes in contact with it whether or not the effect is desired. The ability of nitric oxide to debride a wound is further supported by the possible inhibition of collagen expression due to high concentration of exogenous nitric oxide applied to the wound, as seen in FIG. 6 (left panel).

Preferably, after the exposure of the wound to high concentration of nitric oxide gas for a first treatment period (e.g., 5-8 hours per day), the necrotic tissue may be mechanically removed easily and the concentration of nitric oxide gas can be decreased for a second treatment period. The low concentration of nitric oxide gas (e.g., at 5-20 ppm) delivered for the second treatment period may upregulate the expression of collagen mRNA leading to synthesis of new collagen to aid in the closure of the wound. For example, an increased collagen mRNA expression was observed in fibroblasts exposed to 5 ppm of NO. The second treatment period may be for a period 7-16 hours per day. Further, the treatment with high and low concentration of nitric oxide gas can be repeated for several days.

For chronic non-healing ulcers on the skin, it is also possible to graft natural skin tissue or synthetically produced skin tissue onto the ulcer after the wound has been prepared. Wound bed preparation may include the reduction of microbial load, debridement, and the management of exudate.

It is believed that the body's natural response to injury is to increase the amount of nitric oxide in order to reduce bacterial count at the injury site, help remove dead cells and then promote healing. As a result, NO is produced not just at the injury site, but also in other parts of the body involved as a result the injury, and as a result, this circulates NO around the body in the blood stream. After a few days of this preparation for healing, the body decreases the nitric oxide it produces to a new level that will promote healing. If a wound fails to heal or becomes infected, the body maintains the circulating nitric oxide at a high level and the wound is then caught with a concentration of nitric oxide that may prevent it from healing. It becomes the “Catch 22” of wound healing. Bathing the injury site to high concentration of nitric oxide gas (e.g., 120 ppm to 400 ppm) sends a message to the body that there is enough nitric oxide at the injury site and therefore the body can shut down the extra production by other cells. This enables the local site to heal while it receives the appropriate supraphysiological concentration of nitric oxide gas to inhibit microbial growth.

### Experimental Example 2

### Safety Studies

In addition to the above study showing no toxicity of in vivo exposure of 200 ppm of nitric oxide gas in an animal model for an open wound, studies to confirm the viability of normal host cells exposed to gNO were performed on fibroblasts, endothelial cells, keratinocytes, alveolar epithelial cells, macrophages, and monocytes, in both flat plate and 3-D growth models for some studies. These experiments looked at viability, proliferation, migration, attachment, expression and tube formation in the appropriate models.

Fibroblast cells obtained from adult patients undergoing elective reconstructive surgery were cultured in Dulbeco's Modified Eagle's Medium (DMEM), supplemented with 10% fetal bovine serum (FBS) and antibiotic-antimycotic preparation and divided into ten 25 cm² vented culture flasks (COSTAR). Four of these flasks (treated group) were exposed to 20 or 200 ppm humidified gNO inside a specialized NO incubation chamber at 37° C. for 24 and 48 hours. The NO exposure chamber was validated prior to the study to eliminate extraneous variables and ensure optimal conditions for fibroblast cell growth. Another four flasks (control group) were placed inside conventional culture incubator and exposed only to ambient humidified air at 37° C. Two flasks were separately harvested and counted as the number of cells at zero time. Following the treatment, fibroblast cells were harvested and evaluated for morphology, cell count, capacity to proliferate and medium pH. The results from these experiments show that exposure to around 200 ppm of gNO did not have harmful effects on the fibroblast.

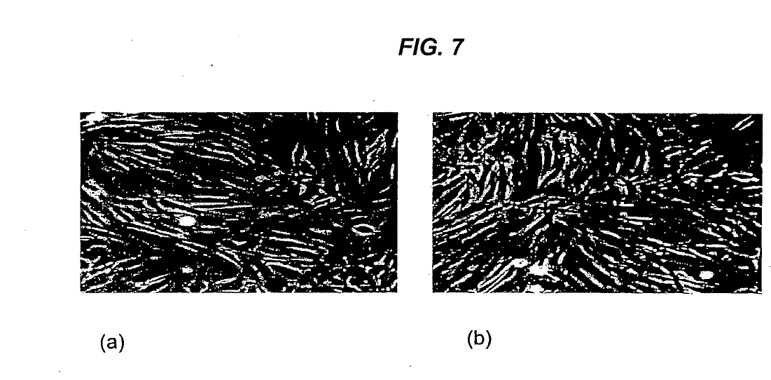

FIG. 7 shows morphology of fibroblast cells from the viability study, where cultured human fibroblast cells were exposed to various gNO concentrations less than 200 ppm continuously for 48 hours. Morphological appearance and attachment capacity of control and treated dermal fibroblasts cells following 48 hours period were quite comparable. Cells under gNO appeared healthy and attached to the culture plates. No toxic effect due to exposure to gNO was observed.

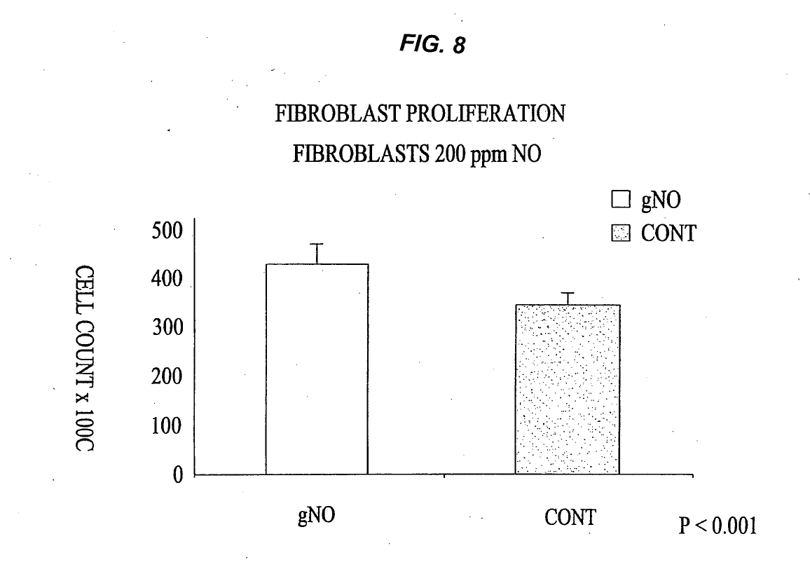

FIG. 8 shows that, in addition to a lack of toxicity to fibroblast cells, exposure to 200 ppm NO may also have positive effect of increasing proliferation of fibroblast cells that may further aid in the wound healing process. It will also be understood, in view of FIG. 8, that exposure to 300 ppm NO may also have positive effect of increasing proliferation of fibroblast cells that may further aid in the wound healing process.

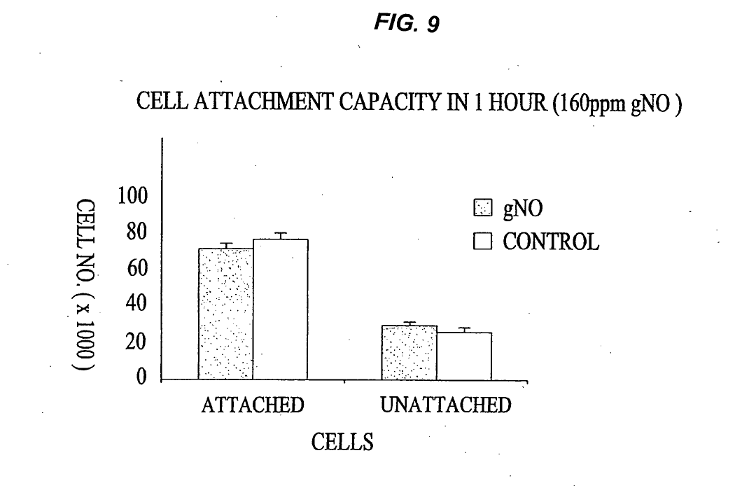

FIG. 9 shows results from cell attachment capacity from the fibroblast cells exposed to 160 ppm of gNO. Capability of cells to reattach to the culture plates within a specified time limit is commonly used as an indication of viability of cells in culture. Both the control and treated groups show a 70% attachment capacity within 1 hour of culturing. This result in conjunction with cell morphology and count support the safety of gNO therapy for topical applications on mammalian skin tissue at least at a range between 100 to 200 ppm of gNO, and more preferably, at a range between 100 and 300 ppm of gNO.

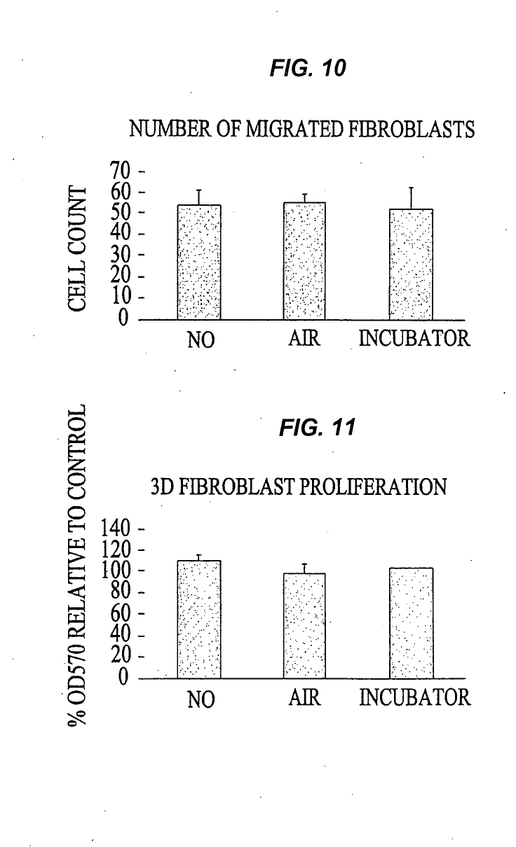

FIG. 10 shows the amount of migration of fibroblasts grown in a 3D matrix and exposed to 200 ppm NO for 8 hours per day for 3 days compared with control cells in air or conventional incubator. As seen from these results, NO does not appear to affect (or more specifically does not interfere with) the migration of these fibroblasts under these conditions.

FIG. 11 shows the amount of proliferation of fibroblasts grown in a 3D matrix and exposed to 200 ppm NO for 8 hours per day for 3 days compared with control cells in air or conventional incubator. Again, NO does not appear to interfere with the proliferation of fibroblasts under these conditions.

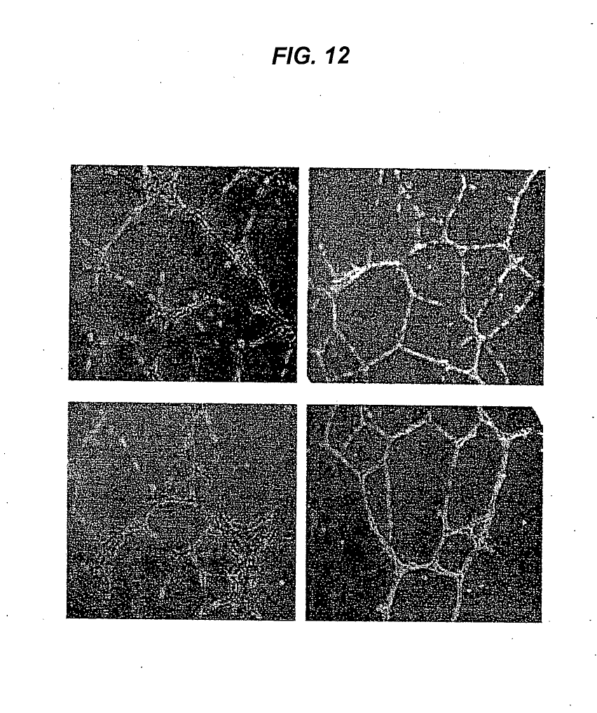

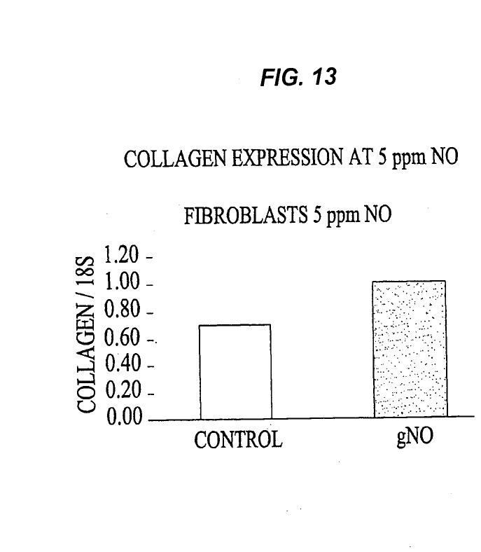

FIG. 12 shows the tube formation in human endothelial cells grown in matrigel and exposed to air (top panels) or 200 ppm NO (bottom panels) for 8 hours (left panels) or 24 hours (right panels). Again, no significant difference between exposure to air and 200 ppm can be discerned.

### Experimental Example 3

### Autoimmune Wound Patient

A patient, a 22 year old female, was diagnosed with antiphospholipid antibody syndrome. Antiphospholipid antibody syndrome is an autoimmune disorder that mainly affects people under the age of 40. The disease causes the blood in the body to clot and is associated with a type of antibody that attacks blood platelets. Strokes, deep vein thrombosis (DVT), heart attacks, and pulmonary emboli are some of the manifestations of the disease. As a result of this syndrome, the patient had two DVT's and one pulmonary embolism.

Following repetitive injury to the deep venous system, the patient developed three areas of ulceration and secondary skin breakdown on the left shin area of the leg. The wound was colonized with a variety of organisms and at times had been clinically infected. The patient had been treated with numerous types of oral and IV antibiotics over several years. Many different types of dressings had been used to control the bacteria, including silver dressings, all without success.

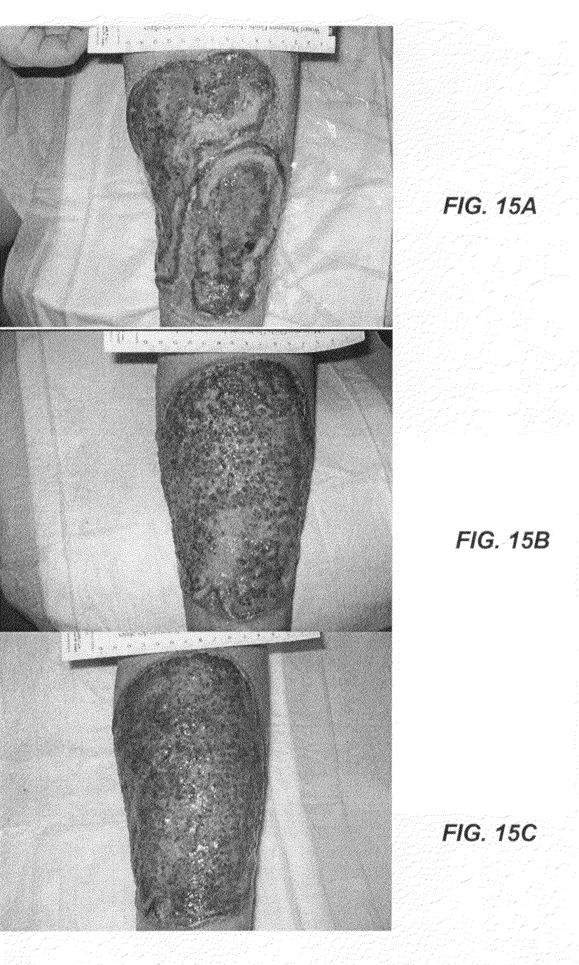

The three small ulcers continued to grow in size and then joined together to create one large ulcer (FIG. 15A). Skin grafts on two occasions deteriorated and sloughed due to repetitive infections and high bacterial burden, even with the use of hyperbaric oxygen treatment.

With the inability to control the bacterial burden, amputation was planned. As a rescue trial, topical nitric oxide gas was applied for eight hours per night. Within six weeks it was noted that there was less yellow slough in the wound bed, the wound edges appeared less raised, and more granulation buds were present (FIG. 15B).

The nitric oxide therapy was then changed to be used for 12 hours per night, using 200 ppm gNO. The wound evaluation following another eight weeks of treatment demonstrated that the wound bed was full of beefy red granulation tissue (covering ˜75% of the wound) (FIG. 15C). The amputation was avoided and the patient was considered eligible for another skin graft.

### Example 4

### Psoriasis Study

Experiments were conducted using one female volunteer, age 28, who suffers from psoriasis. The subject had psoriasis on her arms, legs and feet, all of which is itchy and painful. At the time of the study, the volunteer was using a medicated cream called DOVOBET on the arms and legs. The subject stopped using the medicated cream on the legs and feet prior to the NO treatments described herein.

Treatments were administered to the subject's right foot, as it appeared to have been affected the most by psoriasis. The right side of the foot had a white scaly appearance as well as red inflammation on and around the ankle. Around the ankle were small red blotches that spread all of the way up the leg. The small blotches were slightly raised and appeared to be inflamed.

The subject's foot was placed into a large plastic boot, which was lightly secured with VELCRO straps halfway up the calf. A tube coming out of the top of the plastic boot was unaltered, and the second tube was sealed off using a plastic clamp. A NO line capable of delivering NO at 300 ppm, 250 cc per minute, was screwed into the bottom tubing closest to the ankle using a plastic screw. A pin was used to pick several holes in the bottom of the plastic boot to allow the NO to flow through the boot. NO was then administered to the boot, and therefore, the psoriatic lesions, at 300 ppm for 180 minutes per day over six consecutive days. Table 1 illustrates the course of treatment for the subject.

TABLE 1
Courses of treatment for treatment of psoriasis in a subject.

| Day | NO Concentration | Time |
|---|---|---|
| 1 | 300 ppm | 180 min |
| 2 | 300 ppm | 180 min |
| 3 | 300 ppm | 180 min |
| 4 | 300 ppm | 180 min |
| 5 | 300 ppm | 180 min |
| 6 | 300 ppm | 180 min |

During the six days of testing and treatment of the subject, there was an additional result. A couple of days into testing, it was observed that on the treated leg, where a dark patch of skin located on the inner calf was always located, a change began to occur. The subject noted that all the psoriasis creams previously used never altered the coloring of the discolored patch of skin, but after day two of the NO treatments set forth herein, the discolored patch was almost gone (i.e., the discoloration was eliminated and the area where the patch was previously observed was now the same color as all the rest of the skin on the leg).

After the first day of NO treatment, the white scales on the ankle disappeared. The white scales would reappear to a lesser degree on the next day, prior to the next course of NO treatment.

After the first treatment of 300 ppm NO (day one), no change was observed in the appearance of the psoriasis.

After the second treatment of 300 ppm NO (day two), the inflammation around the ankle appeared to have diminished slightly. The small red blotches around the ankle and extending up the leg appeared to be the same as prior to the treatment.

After the third treatment of 300 ppm NO (day three), the psoriatic lesion around the ankle appeared to be smaller in area, and the inflammation had decreased considerably. The small red blotches around the ankle and extending up the leg appeared the same.

After the fourth, fifth and sixth treatments of 300 ppm NO (days 4, 5 and 6), the physical results appeared to remain the same, with no further advancement of disease state, and no further improvement of the psoriatic condition.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of using topical nitric oxide exposure to treat tissue affected by an autoimmune disorder, comprising providing a source of nitric oxide containing gas, delivering the nitric oxide containing gas to a skin surface containing tissue affected by said autoimmune disorder so as to bathe the tissue affected by said autoimmune disorder with nitric oxide, and optionally, evacuating the nitric oxide containing gas from the area surrounding the tissue affected by said autoimmune disorder.

2. The method of claim 1, wherein said autoimmune disorder is a skin-related autoimmune disorder.

3. The method of claim 1, wherein the nitric oxide is delivered to the skin surface using a bathing unit surrounding the tissue affected by said autoimmune disorder.

4. The method of claim 1, wherein the concentration of nitric oxide in the nitric oxide containing gas that bathes the tissue affected by said autoimmune disorder is equal to or less than about 300 ppm.

5. The method of claim 1, further comprising the step of refreshing the tissue affected by said autoimmune disorder with a fresh supply of nitric oxide containing gas.

6. The method of claim 1, wherein a jet of nitric oxide containing gas is delivered to the portion of the skin surface containing the tissue affected by said autoimmune disorder.

7. The method of claim 1, wherein the source of nitric oxide containing gas is selected from the group consisting of a pressurized cylinder containing nitric oxide gas and direct production of nitric oxide gas.

8. The method of claim 1, further comprising at least one of the steps selected from the group consisting of:
a. monitoring the concentration of nitric oxide bathing the tissue affected by said autoimmune disorder;
b. monitoring the concentration of nitrogen dioxide bathing the tissue affected by said autoimmune disorder;
c. stripping nitric oxide from the evacuated gas; and
d. stripping nitrogen dioxide from the evacuated gas.

9. The method of claim 2, wherein the skin-related autoimmune disorder is selected from the group consisting of alopecia areata, psoriasis, vitiligo, epidermolysis bullosa, scleroderma, antiphospholipid syndrome, Sjorgren's Syndrome and Raynaud's Syndrome.

10. The method of claim 1, further comprising the step of evacuating the nitric oxide containing gas from the area surrounding the tissue affected by said autoimmune disorder.

11. The method of claim 5, wherein the step of refreshing utilizes an agitator.

12. A method of treating skin tissue affected by a skin-related autoimmune disorder comprising the steps of: identifying the affected skin tissue of a human; providing a flow-controlled source of nitric oxide gas; and delivering the nitric oxide gas to at least a portion of the affected skin tissue.

13. The method of claim 12 further comprising the step of sealing the nitric oxide gas to prevent contact with air in the atmosphere.

14. The method of claim 12 further comprising the step of diluting the nitric oxide gas with at least one of the members selected from the group consisting of air and an inert gas.

15. The method of claim 12 wherein the flow-controlled source of nitric oxide gas flows from a pressurized cylinder containing nitric oxide gas.

16. The method of claim 15 further comprising the step of reducing pressure of the nitric oxide gas before delivering the nitric oxide gas to the affected skin tissue.

17. The method of claim 12 further comprising evacuating the nitric oxide gas at a flow rate substantially equal to a flow rate of the nitric oxide gas delivered to the affected skin tissue.

18. A method of treating skin tissue affected by an autoimmune disorder with topical exposure to nitric oxide gas comprising the steps of: providing a source of nitric oxide gas; diluting the nitric oxide gas with at least one of the members selected from the group consisting of air and an inert gas; and delivering the diluted nitric oxide gas to at least a portion of the affected skin tissue.

19. The method of claim 18 wherein the step of diluting the nitric oxide gas is performed through a gas blender.

20. The method of claim 18 wherein the nitric oxide gas delivered to the skin is flow controlled.

21. The method of claim 18 wherein the step of diluting the nitric oxide gas further comprises the step of delivering the inert gas to a substantially air-tight sealed area over the affected skin tissue.

22. The method of claim 18 further comprising the step of monitoring the concentration of nitric oxide being delivered.

23. The method of claim 3, wherein said bathing unit comprises one or more openings for the escape of gas.

24. A method of alleviating Alopecia areata-mediated loss of hair on a skin surface, said method comprising:
a. providing a source of nitric oxide containing gas; and
b. delivering said nitric oxide containing gas to said skin surface which is afflicted with hair loss so as to bathe said skin surface with nitric oxide,
thereby alleviating said loss of hair from said skin surface.

25. A method of alleviating autoimmune-mediated scarring of a skin surface, wherein said scarring is a result of excess collagen deposition, said method comprising:
a. providing a source of nitric oxide containing gas; and
b. delivering said nitric oxide containing gas to said skin surface which is afflicted with scarring so as to bathe said skin surface with nitric oxide,
thereby alleviating said scarring of said skin surface.
